(19) United States
(12) Patent Application Publication
(10) Pub. No.: US 2012/0321637 A1
(43) Pub. Date: Dec. 20, 2012

(54) COMBINATION CANCER THERAPY WITH HERV INHIBITION

(75) Inventors: Jianli Dong, League City, TX (US); Gengming Huang, Galveston, TX (US)

(73) Assignee: The Board of Regents of the University of Texas System, Austin, TX (US)

(21) Appl. No.: 13/525,396

(22) Filed: Jun. 18, 2012

Related U.S. Application Data
(60) Provisional application No. 61/498,887, filed on Jun. 20, 2011.

(51) Int. Cl. A61K 39/42; A61K 31/44; A61K 31/4439; A61K 31/506; A61K 31/517; A61K 31/5377; A61K 31/4523; A61K 31/4025; A61K 31/519; A61K 31/7072; A61K 31/7068; A61K 31/513; A61K 31/551; A61K 31/536; A61K 39/395; A61P 35/00; A61P 31/12; A61K 31/437

(52) U.S. Cl. 424/142.1; 514/300; 514/346; 514/338; 514/252.19; 514/266.4; 514/233.8; 514/252.18; 514/266.24; 514/210.18; 514/275; 514/422; 514/264.11; 514/252.16; 424/147.1; 514/50; 514/45; 514/49; 514/274; 514/220; 514/230.5

(57) ABSTRACT

Embodiments are directed to compositions and methods related combination therapy with HERV inhibition.

### STATEMENT REGARDING PRIORITY

This Application claims priority to U.S. Provisional Patent Application No. 61/498,887 filed Jun. 20, 2011, which is incorporated herein by reference in its entirety.

### BACKGROUND

A. Field of the Invention

Embodiments of this invention are directed generally to biology and medicine. Certain aspects are directed to a combination therapy for cancer.

B. Background

Melanoma is one of the most prevalent malignancies with invasive melanoma having a very poor prognosis. Multiple genetic and environmental factors have been linked to the development and aggressive behavior of melanomas. Dysregulated RAS-RAF-MEK-ERK pathway (MEK is the mitogen activated protein kinase/ERK kinase and ERK is extracellular signal regulated kinase) and p16INK4A-cyclin D:cyclin-dependent kinases (CDK) 4/6-retinoblastoma (RB) pathways commonly co-exist and interact functionally in melanoma cells (Rotolo et al., Int J Cancer 115, 164-169, 2005; Zhao et al., Biochem Biophys Res Commun 370, 509-513, 2008; Li et al., Cancer Invest., 2009). BRAF mutations, found in up to 60-80% of melanomas, can upregulate the MAP Kinase pathway. Similarly, a majority of melanomas have a deregulated p16INK4A/CDK4/RB pathway in which there is a loss of normal p16 resulting in disinhibition of CDK4.

Even with the discovery of these cellular mechanisms related to cancer and melanoma, there remains a need for additional therapies for treatment of melanoma and other cancers.

### SUMMARY

The inventors have shown that human endogenous retrovirus type K (HERV-K) protein expression is associated with MEK-ERK and p1 6INK4A-CDK4 pathways in melanoma cells. The inventors contemplated that cells with activated HERV can be resistant to the therapeutic effects of MEK and CDK4 blockers. Certain embodiments are directed to methods of treatment that target MEK, CDK4, and HERV. In certain aspects, targeting HERV can provide additional benefits to MEK and/or CDK4 targeted therapies.

Certain embodiments are directed to methods for treating cancer comprising administering an effective amount of an anti-HERV therapy in combination with an effective amount of an inhibitor of BRAF or MEK (e.g., a small molecule inhibitor), and a small molecule inhibitor of CDK4 (e.g., a small molecule inhibitor) to a patient that has cancer. In certain aspects the cancer is melanoma or breast cancer. In certain aspects the anti-HERV therapy is administered about 1, 2, 3, 4, 5, 6, 7, 8, 9, or 10 hours or days prior to administration of the inhibitors of BRAF-MEK and the inhibitor of CDK4. In a further aspect the anti-HERV therapy is administered simultaneously with a small molecule inhibitor of BRAF or MEK. In still another aspect the anti-HERV therapy is administered simultaneously with a small molecule inhibitor of CDK4. And in still another aspect the anti-HERV therapy is administered simultaneously with both a small molecule inhibitor of BRAF or MEK, and a small molecule inhibitor or CDK4. In certain aspect two or three or more of an anti-HERV, a small molecule inhibitor of BRAF or MEK, and/or a small molecule inhibitor of CDK4 are present in the same pharmaceutical formulation. In other aspects each of the anti-HERV therapy, small molecule inhibitor of BRAF or MEK, and a small molecule inhibitor of CDK4 are in separate pharmaceutical formulations. The pharmaceutical formulations can be administered at approximately the same time (i.e., simultaneously).

In certain aspects the inhibitor of BRAF or MEK is PLX4032 (Plexxikon), BAY 43-9006 (Sorafenib), Raf-265 (CHIR-265), XL281 (Exelixis), dasatinib, erlotinib hydrochloride, gefitinib, imatinib mesilate, lapatinib, sorafenib tosylate, sunitinib malate, PD-325901, XL518, PD-184352, PD-318088, AZD6244, CI-1040, vemurafenib (Zelboraf), GSK2118436, or combinations thereof.

In certain aspects the inhibitor of CDK4 is P-276-00, GW-491619 (GlaxoSmithKine), AG-12275 (Pfizer), AG-12286 (Pfizer); PD-0166285 (Pfizer); or PD-0332991 (Pfizer).

In another aspect, the anti-HERV therapy is combined with a CTLA-4 inhibitor such as ipilimumab (Yervoy), which recently gained FDA approval.

In certain aspects the anti-HERV therapy is a monoclonal antibody that specifically binds a HERV protein or blocks the binding of a HERV protein to a cell surface; and/or an anti-retroviral drug. The anti-HERV monoclonal antibody can specifically bind a HERV envelope protein. In certain aspects the HERV envelope protein is a HERV-K envelope protein. The anti-retroviral is selected from nucleoside reverse transcriptase inhibitors, non-nucleoside reverse transcriptase inhibitors, protease inhibitors, fusion inhibitors, and integrase inhibitors. In a further aspect the anti-retroviral is AZT. In other aspects, HERV-K is inhibited by RNAi.

In certain aspects the anti-retroviral is one or more of AZT, dideoxyinosine (ddI), dideoxycytidine (ddC), LAMIVUDINE (3TC), STAVUDINE (d4T), ABACAVIR (1592U89), ADEFOVIR DIPIVOXIL (bis(POM)-PMEA), NEVIRAPINE (BI-RG-587), DELAVIRDINE (BHAP, U-90152), EFAVIRENZ (DMP 266), INDINAVIR (MK-639), RITONAVIR (ABT-538), SAQINAVIR (Ro-31-8959), NELFINAVIR (AG-1343), AMPRENAVIR (141W94), or combinations thereof.

In certain embodiments the cancer is melanoma or breast cancer. In certain aspects the melanoma is premalignant, malignant, metastatic, or drug-resistant.

Certain embodiments are directed as treating patient having an intrinsic or acquired resistance to an anti-cancer therapy. Intrinsic resistance is when the cancer cell prior to any treatment is resistant to a cancer therapy. Acquired resistance is when a cancer is initially susceptible to a therapy and develops a resistance to the therapy once therapy has been administered. In certain aspects, an anti-HERV agent can be used to sensitize a cancer to one or more anti-cancer agents, such as a BRAF or MEK inhibitor, a CDK4 inhibitor, an anti-CTLA-4 agent, or other known cancer or melanoma therapy.

A further embodiment is directed to an anti-cancer composition comprising an anti-retroviral, an inhibitor of BRAF or MEK, and an inhibitor of CDK4.

As used herein, an “inhibitor” can be any chemical compound, peptide, or polypeptide that can reduce the activity or function of a protein. An inhibitor as provided by the invention, for example, can inhibit directly or indirectly the activity of a protein. Direct inhibition can be accomplished, for example, by binding to a protein and thereby preventing the protein from binding an intended target, such as a receptor, or by inhibiting an enzymatic or other activity of the protein, either competitively, non-competitively, or uncompetitively. Indirect inhibition can be accomplished, for example, by binding to a protein's intended target, such as a receptor or binding partner, thereby blocking or reducing activity of the protein.

The term “effective amount” means an amount effective, at dosages and for periods of time necessary, to achieve the desired therapeutic or prophylactic result.

An “effective amount” of an anti-cancer agent in reference to decreasing cancer cell growth, means an amount capable of decreasing, to some extent, the growth of some cancer or tumor cells. The term includes an amount capable of invoking a growth inhibitory, cytostatic and/or cytotoxic effect and/or apoptosis of the cancer or tumor cells.

A “therapeutically effective amount” in reference to the treatment of cancer, means an amount capable of invoking one or more of the following effects: (1) inhibition, to some extent, of cancer or tumor growth, including slowing down growth or complete growth arrest; (2) reduction in the number of cancer or tumor cells; (3) reduction in tumor size; (4) inhibition (i.e., reduction, slowing down, or complete stopping) of cancer or tumor cell infiltration into peripheral organs; (5) inhibition (i.e., reduction, slowing down, or complete stopping) of metastasis; (6) enhancement of anti-tumor immune response, which may, but is not required to, result in the regression or rejection of the tumor, or (7) relief, to some extent, of one or more symptoms associated with the cancer or tumor. The therapeutically effective amount may vary according to factors such as the disease state, age, sex and weight of the individual and the ability of one or more anti- cancer agents to elicit a desired response in the individual. A “therapeutically effective amount” is also one in which any toxic or detrimental effects are outweighed by the therapeutically beneficial effects.

The phrases “treating cancer” and “treatment of cancer” mean to decrease, reduce, or inhibit the replication of cancer cells; decrease, reduce or inhibit the spread (formation of metastases) of cancer; decrease tumor size; decrease the number of tumors (i.e. reduce tumor burden); lessen or reduce the number of cancerous cells in the body; prevent recurrence of cancer after surgical removal or other anti-cancer therapies; or ameliorate or alleviate the symptoms of the disease caused by the cancer.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Quantification of human genome integrated HERV-K. Genomic DNA is extracted from melanoma cells and subjected to Alu-HERV-K GAG PCR. The 1^{st} round PCR is genome-anchored by 5′ primer using human Alu sequence thereby amplifying only genome-integrated HERV-Ks. Amplicons from 1^{st} round PCR are subjected to 2^{nd} PCR amplification using nested HERV-K GAG primers. The 2^{nd} round PCR amplicons are quantified by real-time PCR.

FIGS. 2A-2B. HERV-K ENV is expressed in polyploid cells. Formalin-fixed, paraffin embedded microscopic sections of nevus (A) and melanoma (B) were analyzed by immuno-staining using HERV-K ENV specific antibody. A representative staining pattern is shown. Note the significant numbers of polyploid cells in the melanoma but not in the nevus. Also, note that all such cells expressed ENV (B). Magnification: ×100.

FIG. 3. HERV-K inhibition blocks intercellular fusion mediated colony formation. Human melanoma cell line A375 was transiently transfected and selected to stably express either control pS-puro-scrambled or HERV-K shRNA pS-puro-H-Ki construct; both carry puromycin selection marker, or pEYFP-neo-N3 (Clontech) that carries yellow fluorescent protein (YFP) with a neomycin selection marker. To examine cell fusion, 10⁶ of A375 cells stably expressing pEYFP-N3 were mixed with equal numbers of the cells expressing either pS-puro-scrambled (A) or pS-puro-H-Ki (B). Mixed cells were cultured to confluence in media without puromycin or G418 selection. Cells were then trypsinized and passaged in culture medium that includes both puromycin and G418 to select pS-puro-scrambled-pEYFP-N3 and pS-puro-H-Ki-pEYFP-N3 fused cells. Colony formation assay was used to count “fused” clones under double selection.

FIG. 4. Inhibition of cell fusion using HERV-K ENV monoclonal antibodies. 10⁴ A375S cells (stably transfected with pS-puro-scrambled) and 10⁴ A375Y cells (stably transfected with pEYFP-N3-neo) were added per well in 96 well plate. Different dilutions of HERV-K monoclonal antibodies HERM-1811-5 (immunized with 79.5 kDa full length HERV-K ENV) or HERM-1821-5 (immunized with 42.8 kDa C-terminal region of HERV-K ENV) were added to culture media. PBS solvent was used as control. Cells were cultured for 48 h, followed by selection of pS-puro-scrambled-pEYFP-N3 fused cells using 1 μg/ml puromycin and 700 μg/ml G418 for 48 hours. Syncytia ratio was determined by the ratio of total cell numbers in the antibody treated over PBS control group. Since the selective medium will only allow cells to survive that have fused to allow complementation of the necessary proteins, the total cells remaining are the results of fusion events.

FIG. 5. Depicts various pathways in the cell that are responsible for cell proliferation. PD98059 and 219476 are commercially available inhibitors of MEK and CDK4, respectively.

FIG. 6. Proposed role of HERV sequences in melanoma progression.

FIG. 7. The A101D cell line was treated with various combinations of PD98059 (P), 219476 (C), and AZT (A) for 48hrs after which the MTS assay was done. Triple treatment (P-C-A) resulted in the lowest viability at 51%. Double treatment (P-C) also resulted in a reduction to around 40%. AZT alone achieved a reduction in viability to 87.4%.

FIG. 8. The 624Mel cell line was also treated with the same combinations and concentration of treatments. Triple treatment, inhibiting cell proliferation to 49% was significantly less than double treatments or monotherapies. AZT as part of double treatments showed less reduction in viability than the P-C combination.

### DESCRIPTION

Melanoma is the most lethal skin malignancy, notorious for aggressive growth and resistance to therapy. A phase I clinical trial with PLX4032, a specific inhibitor of mutant BRAF, has generated great excitement because approximately 80% of BRAF mutant metastatic melanomas regressed in response to PLX4032 treatment. Though this trial was considered a true victory in the fight against melanomas, attention has been drawn to the fact that regressed tumors may resurge more aggressively within 8 months after the start of therapy, indicating that more research is needed to conquer melanoma.

Constitutive deregulation of BRAF-MEK-ERK and p16INK4A-CDK4-RB pathways both occur at high frequencies in melanomas. The inventors have shown that suppression of either BRAF-MEK or CDK4 inhibits cell growth, and that simultaneous inhibition of both BRAF-MEK and CDK4 compounds this effect and also triggers significant apoptosis in melanoma cells. The data suggest that BRAF-MEK-ERK and p16INK4A-CDK4-RB pathways may act synergistically in the malignant growth of melanoma cells, and could be jointly targeted for treatment of melanoma. The inventors have also reported that the expression of K type human endogenous retrovirus (HERV-K) correlates with ERK activation and p16INK4A loss in melanoma cells and can be inhibited by MEK and CDK4 inhibitors, especially in combination. Given that HERV-K may drive malignant growth downstream of BRAF-MEK and CDK4, and can be activated by UV and other factors that may be independent of BRAF-MEK and CDK4, the inventors contemplated that cells with activated HERV-K may escape the therapeutic effects of MEK and CDK4 inhibitors, and that triple inhibition of BRAF-MEK, CDK4, and HERV-K would be more effective than double inhibition.

A. Compositions of the Invention

The inventors have shown that HERV-K protein expression is associated with MEK-ERK and p16INK4A-CDK4 pathways in melanoma cells. The inventors contemplated that cells with activated HERV may not respond to the therapeutic effects of MEK and CDK4 blockers and that a treatment comprising triple inhibition of MEK, CDK4, and HERV should be more effective than double treatment.

1. Inhibitors of Human Endogenous Retrovirus (HERV)

Endogenous retroviruses (ERVs) are sequences in the genome thought to be derived from ancient viral infections of germ cells in humans, mammals, and other vertebrates; as such their proviruses are passed on to the next generation and now remain in the genome.

Retroviruses are single-stranded RNA viruses that reverse-transcribe their RNA into DNA for integration into the host's genome. Most retroviruses (such as HIV-1) infect somatic cells, but in very rare cases, it is thought that exogenous retroviruses have infected germline cells (cells that make eggs and sperm) allowing integrated retroviral genetic sequences to be passed on to subsequent progeny, thereby becoming ‘endogenous’. Endogenous retroviruses have persisted in the genome of their hosts for thousands of years. However, they are generally only infectious for a short time after integration as they acquire many inactivating mutations during host DNA replication. They can also be partially excised from the genome by a process known as recombinational deletion. Some human ERVs have been implicated in certain autoimmune diseases and cancers, such as rheumatoid arthritis, teratocarcinoma, melanoma, and breast cancer.

HERVs can be classified to over 20 families based on tRNA specificity of the primer binding site used to initiate reverse transcription. HERV sequences are similar to the HIV sequence. HERV-K activation was correlated with changes in growth characteristics of melanoma cells (e.g., changes in cell shape, loss of melanin, anchorage- independent growth) (Serafino et al., Exp Cell Res 315, 849-862, 2009). There are many thousands of endogenous retroviruses within human DNA, with HERVs comprising nearly 8% of the human genome and composed with 98,000 elements and fragments. HERVs are typically not capable of replication and appeared to be defective in some aspect. The HERV-K family make up less that 1% of the HERV elements. It has been shown that HERV-K is expressed in melanoma cells but not in melanocytes.

It has been hypothesized that HIV induces HERV expression in HIV infected cells, and that a vaccine targeting HERV antigens could therefore specifically eliminate HIV infected cells. The potential advantage of this novel approach is that, by using HERV antigens as surrogate markers of HIV infected cells, it could circumvent the difficulty inherent in directly targeting notoriously diverse and rapidly mutating HIV antigens.

Thus, HERV can be and is a known drug target and can be targeted by anti-retroviral drugs. A wide variety of anti-retroviral drugs are known. Many small molecule (e.g. organic compounds) and macromolecule (antisense DNAs/RNAs, ribozymes, viral surface protein-binding proteins or nucleotides, etc.) drugs have been developed to treat the HIV retrovirus. These same drugs can be used to inhibit HERV. Many drugs have been developed to target critical enzymes of retroviruses and inhibit replication of the virus inside the host cell. For example, nucleoside or nucleotide analogs such as AZT, dideoxycytidine (ddC), and dideoxyinosine (ddI) were developed to inhibit reverse transcriptase (RT) of retroviruses by acting as competitive inhibitors and chain terminators. Non-nucleoside or nucleotide inhibitors have also been found to inhibit reverse transcriptase activity of retroviruses by exerting an allosteric effect by binding to a hydrophobic pocket close to the active site of RT. The protease (PRO) inhibitors in current use are targeted at the active site of the enzyme.

In addition to the RT and PRO inhibitors, other classes of antiviral agents targeting different components of retroviruses or interfering with different stages of retroviral life cycle may be used in conjunction with BRAF-MEK and CDK4 inhibitors. Synthetic peptides and antibodies have been shown to inhibit retrovirus infection by disrupting the function of envelope proteins.

In the pharmaceutical compositions of the present invention nucleoside reverse transcriptase inhibitors, non-nucleoside reverse transcriptase inhibitors, protease inhibitors, fusion inhibitors and integrase inhibitors can be used in combination with BRAF-MEK and CDK4 inhibitors. Examples of the nucleoside reverse transcriptase inhibitor include, but are not limited to ZIDOVUDINE (AZT), DIDANOSINE (ddI), ZALCITABINE (ddC), LAMIVUDINE (3TC), STAVUDINE (d4T), ABACAVIR (1592U89), and ADEFOVIR DIPIVOXIL (bis(POM)-PMEA). Examples of non-nucleoside HIV reverse transcriptase inhibitors include, but are not limited to NEVIRAPINE (BI-RG-587), DELAVIRDINE (BHAP, U-90152) and EFAVIRENZ (DMP 266). Examples of protease inhibitors include, but are not limited to INDINAVIR (MK-639), RITONAVIR (ABT-538), SAQINAVIR (Ro-31-8959), NELFINAVIR (AG-1343), and AMPRENAVIR (141 W94).

The pharmaceutical compositions of the present invention include BRAF-MEK and CDK4 inhibitors in combination with one or more antiretroviral drugs, preferably with a “cocktail” of nucleoside reverse transcriptase inhibitors, non-nucleoside reverse transcriptase inhibitors, and protease inhibitors. For example, BRAF-MEK and CDK4 inhibitors may be combined with two nucleoside reverse transcriptase inhibitors (e.g. ZIDOVUDINE (AZT) and LAMIVUDINE (3TC)), and one protease inhibitor (e.g. INDINAVIR (MK-639)).

2. BRAF or MEK (BRAF-MEK) Inhibitors

An important signaling pathway in melanoma is the RAS/RAF mitogen-activated protein kinase (MAPK)/extracellular signal-regulated kinase (ERK) kinase (MEK)/ERK cascade. This signaling pathway leads to the phosphorylation of several cytosolic and nuclear proteins to regulate gene expression, and thus plays a critical role in cell proliferation, differentiation, senescence and survival. One of the three RAF isoforms in humans, BRAF, is mutated in 50% to 70% of melanoma cases. The ERK pathway is therefore hyperactive in most melanoma cases. A number of small molecule inhibitors that target BRAF, or its downstream effector, MEK, are in clinical and preclinical development. Sorafenib for example, is a broad specificity kinase inhibitor that targets BRAF, CRAF, and receptor tyrosine kinases. Sorafenib is 4-[4-[[4-chloro-3-(trifluoromethyl)phenyl]carbamoylamino]phenoxy]-N-methyl-pyridine-2-carboxamide and is described in U.S. Pat. No. 7,235,576, which is incorporated herein by reference in its entirety. When used as a monotherapy, sorafenib only shows marginal clinical benefit in melanoma patients. Combining drugs that target BRAF or MEK with an agent that inhibits the rescue pathways stimulated by TNF-alpha provides a rational approach to treating melanoma.

Examples of therapeutic agents that target BRAF include, dasatinib, erlotinib, gefitinib, imatinib, lapatinib, Sorafenib, and sunitinib, or a derivative thereof. Preferentially, the derivative of the BRAF inhibitor may be a salt. Thus, according to the invention the BRAF inhibitor may be selected from the group consisting of BAY 43-9006 (Sorafenib), Raf-265 (formerly CHIR-265, Novartis), PLX-4032 (Plexxikon) and XL281 (Exelixis), dasatinib, erlotinib hydrochloride, gefitinib, imatinib mesilate, lapatinib, sorafenib tosylate, and sunitinib malate.

Thus, according to the invention the MEK inhibitor may be selected from the group consisting of certain experimental compounds, some of which are currently in Phase I or Phase II studies, namely PD-325901 (Phase I), XL518 (Phase I), PD-184352, PD-318088, AZD6244 (Phase II) and CI-1040.

Descriptions of MEK inhibitors can be found in U.S. Pat. Nos. 5,525,625, 6,251,943, 6,310,060, 6,638,945, 6,440,966, 6,455,582, 6,496,004, 6,506,798, 6,638,945, 6,696,440, 6,770,778 B2, and 6,809,106 B1; published U.S. Patent Application Nos. 2003/0078428, 2003/0125359, 2003/0216460, 2003/0225076, 2004/0232869, 2004/0054172, and 2004/0006245; published PCT application nos. WO 00/68201; WO 00/68200; WO 00/68199; WO 01/68619, WO 02/076496; WO 03/047585; WO 03/053960, WO 03/062189, WO 03/062191, and WO 03/077914; each of which is incorporated herein by reference.

3. Cdk4 Inhibitors

In certain embodiments of the invention, a Cdk4 inhibitor can be a small molecule. Such inhibitors may be known in the art or as described herein. Non-limiting examples of small molecule Cdk inhibitors include but are not limited to olomoucine, butyrolactone, certain flavonoids, staurosporine and its related compound UCN-01, suramin, toyocamycin, certain ellipticines, certain paullones and certain pyridopyrimidines (Ortega et al., 2002, Biochim Biophys Acta. 1602:73-87; Walker. 1998, Curr Top Microbiol Immunol. 227:149-165; and Garrett & Fattaey. 1999, Curr Opin Genet Dev. 9:104-111). All these compounds have broad spectra against multiple Cdk proteins and other protein kinases. Compounds that are relatively more specific inhibitors of Cdk4 include a triaminopyrimidine derivative CINK4 (Soni et al., 2001, J Natl Cancer Inst. 93:436-446), PD0183812 (Fry et al., 2001, J Biol Chem. 276:16617-16623) and AG12275 (Tetsu and McCormick, 2003, Cancer Cell. 3:233-245; and Toogood, 2001, Med Res Rev. 6:487-498). CDK4 inhibitors include, but are not limited to P-276-00, GW-491619 (GlaxoSmithKine), AG-12275 (Pfizer), AG-12286 (Pfizer); PD-0166285 (Pfizer); or PD-0332991 (Pfizer).

Descriptions of CDK4 inhibitors can be found in: U.S. Pat. Nos. 4,900,727, 5,733,920, 5,849,733, 6,040,321, 6,150,359, 6,262,096, 6,498,163, 6,569,878, 6,593,326, 6,630,464, 6,720,332, 6,756,374, 7,109,229; published U.S. Patent Application Nos. 2002/0151554A1, 2003/0149001, 2003/0087923, 2003/0203907, 2003/0229026, and 2004/0048915; and published PCT applications WO 98/49146, WO 00/12485, WO 01/14375, WO 01/44147, WO 02/20524, and WO 02/28861; each of which is incorporated herein by reference.

B. Pharmaceutical Formulations and Administration

In certain embodiments, the invention also provides compositions comprising 1, 2, 3, or more anti-cancer agents with one or more of the following: a pharmaceutically acceptable diluent; a carrier; a solubilizer; an emulsifier; a preservative; and/or an adjuvant. Such compositions may contain an effective amount of at least one anti-cancer agent. Thus, the use of one or more anti- cancer agents that are provided herein in the preparation of a pharmaceutical composition of a medicament is also included. Such compositions can be used in the treatment of a variety of cancer s. In certain embodiments the treatment is for melanoma or breast cancer.

The anti-cancer agents may be formulated into therapeutic compositions in a variety of dosage forms such as, but not limited to, liquid solutions or suspensions, tablets, pills, powders, suppositories, polymeric microcapsules or microvesicles, liposomes, and injectable or infusible solutions. The preferred form depends upon the mode of administration and the particular disease targeted. The compositions also preferably include pharmaceutically acceptable vehicles, carriers, or adjuvants, well known in the art.

Acceptable formulation components for pharmaceutical preparations are nontoxic to recipients at the dosages and concentrations employed. In addition to the anti-cancer agents that are provided, compositions may contain components for modifying, maintaining, or preserving, for example, the pH, osmolarity, viscosity, clarity, color, isotonicity, odor, sterility, stability, rate of dissolution or release, adsorption, or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin, or immunoglobulins); coloring, flavoring, and diluting agents; emulsifying agents; hydrophilic polymers (such as polyvinylpyrrolidone);

low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid, or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 18 th Ed., (A. R. Gennaro, ed.), 1990, Mack Publishing Company), hereby incorporated by reference.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which may further include sorbitol or a suitable substitute therefor.

The pharmaceutical composition to be used for in vivo administration is typically sterile. Sterilization may be accomplished by filtration through sterile filtration membranes. If the composition is lyophilized, sterilization may be conducted either prior to or following lyophilization and reconstitution. The composition for parenteral administration may be stored in lyophilized form or in a solution. In certain embodiments, parenteral compositions are placed into a container having a sterile access port, for example, an intravenous solution bag or vial having a stopper pierceable by a hypodermic injection needle, or a sterile pre-filled syringe ready to use for injection.

The above compositions can be administered using conventional modes of delivery including, but not limited to, intravenous, intraperitoneal, oral, intralymphatic, subcutaneous administration, intraarterial, intramuscular, intrapleural, intrathecal, and by perfusion through a regional catheter. Local administration to a tumor in question is also contemplated by the present invention. When administering the compositions by injection, the administration may be by continuous infusion or by single or multiple boluses. For parenteral administration, the anti-metastatic agents may be administered in a pyrogen-free, parenterally acceptable aqueous solution comprising the desired anti-cancer agents in a pharmaceutically acceptable vehicle. A particularly suitable vehicle for parenteral injection is sterile distilled water in which one or more anti-cancer agents are formulated as a sterile, isotonic solution, properly preserved.

Once the pharmaceutical composition of the invention has been formulated it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

If desired, stabilizers that are conventionally employed in pharmaceutical compositions, such as sucrose, trehalose, or glycine, may be used. Typically, such stabilizers will be added in minor amounts ranging from, for example, about 0.1% to about 0.5% (w/v). Surfactant stabilizers, such as TWEEN®-20 or TWEEN®-80 (ICI Americas, Inc., Bridgewater, N.J., USA), may also be added in conventional amounts.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents. Compositions for parental administration are also sterile, substantially isotonic and made under GMP conditions.

For the compounds of the present invention, alone or as part of a pharmaceutical composition, such doses are between about 0.001 mg/kg and 1 mg/kg body weight, preferably between about 1 and 100 μg/kg body weight, most preferably between 1 and 10 μg/kg body weight.

Therapeutically effective doses will be easily determined by one of skill in the art and will depend on the severity and course of the disease, the patient's health and response to treatment, the patient's age, weight, height, sex, previous medical history and the judgment of the treating physician.

Certain embodiments are directed to methods of treating cancer by administering one or more therapeutic agents to a cancer cell. In some methods of the invention, the cancer cell is a tumor cell. The cancer cell may be in a patient. The patient may have a solid tumor. In such cases, embodiments may further involve performing surgery on the patient, such as by resecting all or part of the tumor. Compositions may be administered to the patient before, after, or at the same time as surgery or in combination with one or more other compositions. In additional embodiments, patients may also be administered directly, endoscopically, intratracheally, intratumorally, intravenously, intralesionally, intramuscularly, intraperitoneally, regionally, percutaneously, topically, intrarterially, intravesically, or subcutaneously. Therapeutic compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

Methods of treating cancer may further include administering to the patient chemotherapy or radiotherapy, which may be administered more than one time. Chemotherapy includes, but is not limited to, cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxotere, taxol, transplatinum, 5-fluorouracil, vincristin, vinblastin, methotrexate, gemcitabine, oxaliplatin, irinotecan, topotecan, or any analog or derivative variant thereof. Radiation therapy includes, but is not limited to, X-ray irradiation, UV-irradiation, γ-irradiation, electron-beam radiation, or microwaves. In addition, a cell or patient may be administered a protease or peptidase to increase the production of infectious EEV form of the virus from cells. Moreover, a cell or a patient may be administered a microtubule stabilizing agent, including, but not limited to, taxane, as part of methods of the invention. It is specifically contemplated that any of the compounds or derivatives or analogs, can be used with these combination therapies.

In some embodiments, the cancer that is administered the composition(s) described herein may be a bladder, blood, bone, bone marrow, brain, breast, colorectal, esophagus, gastrointestine, head, kidney, liver, lung, nasopharynx, neck, ovary, pancreas, prostate, skin, stomach, testicular, tongue, or uterus cell. In certain aspects the cancer is melanoma or breast cancer.

### II. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, and are not intended as limitations on the scope of the invention. Changes therein and other uses which are encompassed within the spirit of the invention as defined by the scope of the claims will occur to those skilled in the art.

### Example 1

### Combinatorial Inhibition of BRAF-MEK, CDK4, and HERV-K in Melanoma Cells

Tumor development involves molecular and cellular changes leading to the evolution and selection of aggressive clones. HERV-K encodes unique retroviral proteins including reverse transcriptase (RT) and ENV that may be co-opted by melanoma cells leading to tumor progression and treatment resistance. It has been shown that constitutive BRAF-MEK signaling upregulation can drive aneuploidy in melanocytic cells (Cui et al. Cancer Res 2010, 70(2):675-84). The inventors contemplate that this effect is mediated, at least in part, by HERV-K retrotransposition-mediated mutagenesis. Active HERV-K retrotransposition should increase genomic HERV-K copy numbers that can be measured by real-time quantitative PCR. HERV-K ENV is homologous to syncytin. The inventors contemplate that HERV-K ENV has fusogenic activity that can mediate intercellular fusion of melanoma cells. ENV may anchor tumor cells to target sites through intercellular fusions (melanoma cell as seed, ENV as root, target site as soil). Blocking HERV-K expression may stabilize the chromosome, inhibit cell fusion, and prevent evolution and the selection of more aggressive melanoma cells.

Deregulation of BRAF-MEK-ERK and p16INK4A-CDK4-RB pathways is a common paradigm in cancer biology. Since HERV-K is activated in melanoma cells, its inhibition may be important in melanoma therapy. The inventors have demonstrated that combinatorial inhibition of BRAF-MEK and CDK4 enhances apoptosis in melanoma cells, and the effect is associated with further reduction of phospho-ERK (p-ERK) when INK4A cDNA or CDK4 suppression is included to BRAF-MEK inhibition, suggesting that p16INK4A-CDK4 may participate in the signaling cross-talk and feedback regulation of MEK-ERK. The inventors have also shown that the expression of HERV-K correlates with MEK-ERK activation and loss of p16INK4A in melanoma specimens and can be suppressed by MEK and CDK4 inhibitors in cultured melanoma cells. The inventors contemplate that simultaneous inhibition of BRAF-MEK, CDK4, and HERV-K may broaden the pharmacodynamic profile, achieve target inhibition at lower bio-available drug concentrations, and generate synergistic and long-lasting therapeutic effects.

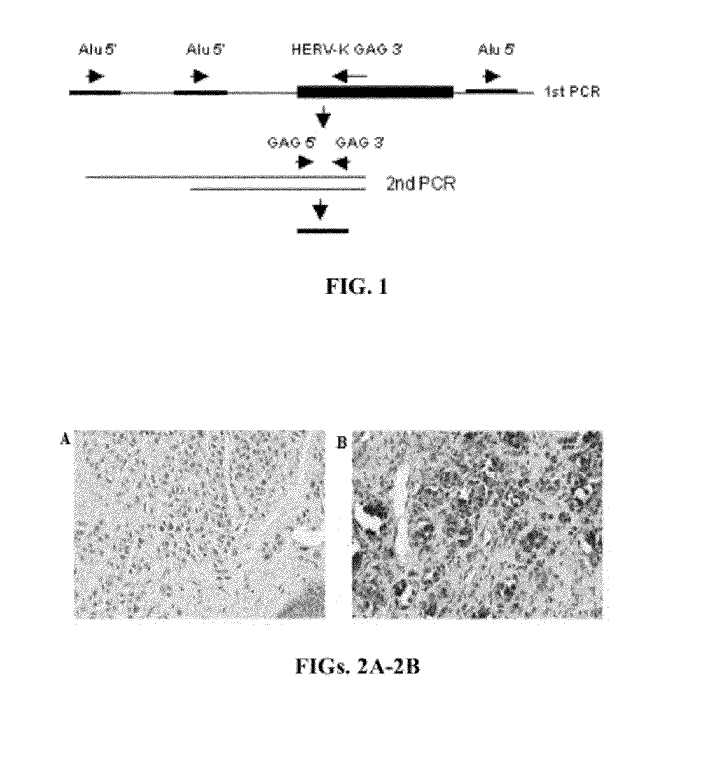

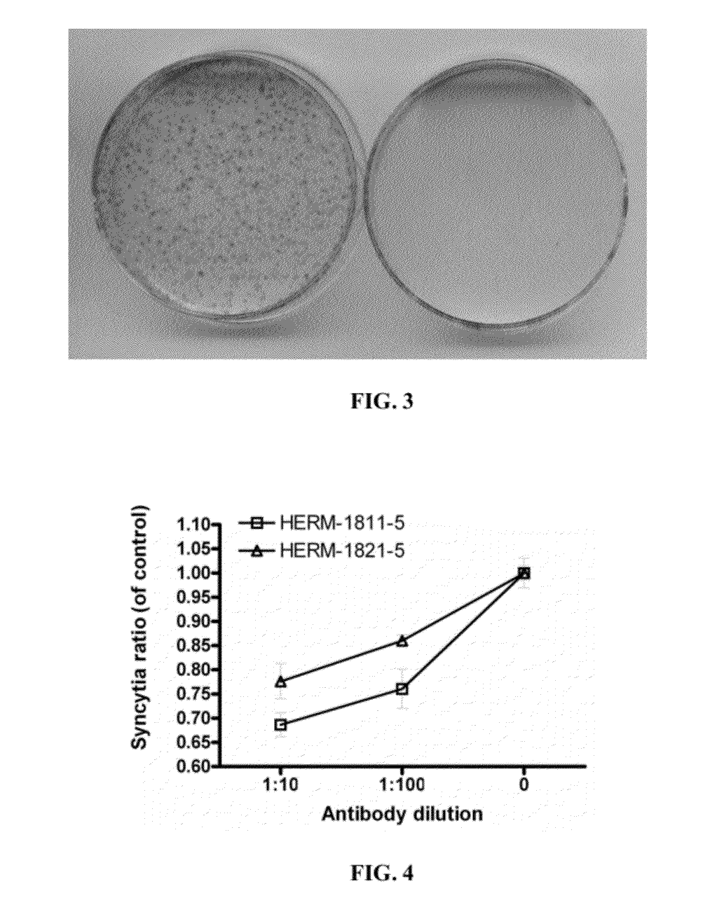

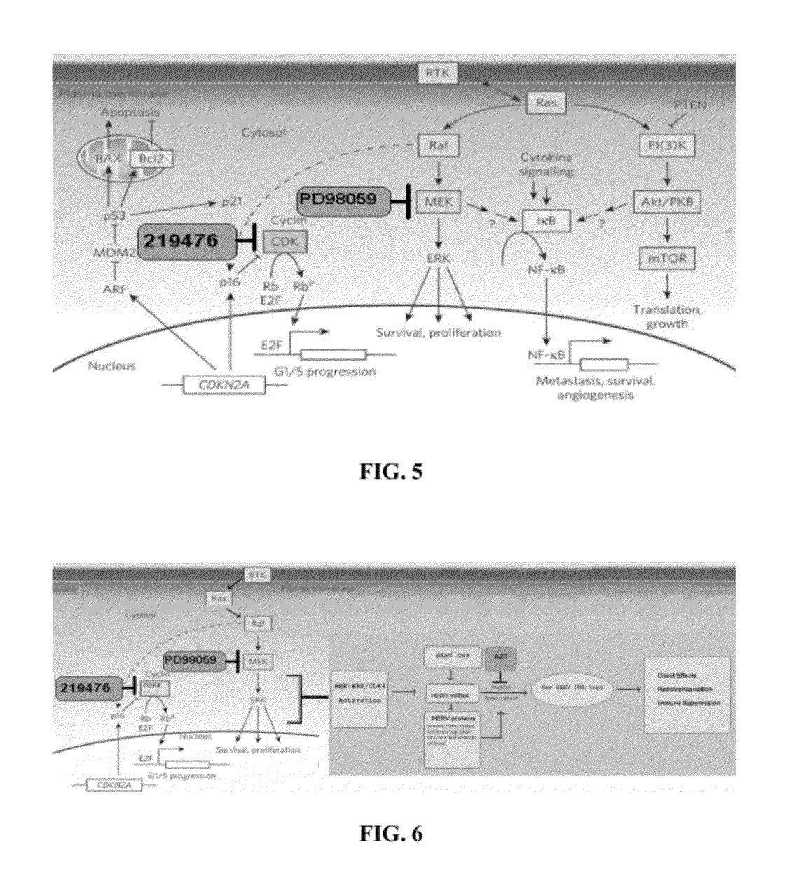

HERV-K activation leads to the transcription of HERV-K RNA and the translation of HERV-K RT, integrase, and other viral proteins. HERV-K RT can reverse transcribe HERV-K RNA back to DNA, and free HERV-K DNA may transpose into host genomic DNA. The inventors designed a long-range (up to 10 kb) nested real-time PCR to quantify genome integrated HERV-K DNA (FIG. 1), using long-range PCR kit (Roche, Indianapolis, Ind., USA) and real-time PCR on a SmartCycler (Bechert et al., Am J Clin Pathol 2010, 133(2):242-50).

HERV-K ENV is expressed in polyploid cells in melanoma specimens. The inventors detected polyploid cells in melanomas but not in nevi (benign chronic lesions of the skin) (FIG. 2), and the melanomas expressed HERV-K ENV (Table 1, FIG. 2). Increased DNA content can be caused by mitosis error or intercellular fusion and is associated with enhanced malignant behavior (Lu and Kang, Cancer Res 2009, 69(22):8536-9; Panigrahi and Pati, Crit Rev Oncol Hematol 2009, 72(3):181-93; Rajaraman et al., Cancer Cell Int 2006, 6:25).

TABLE 1
Expression of HERV-K EVE in Binuclear and Multinuclear Cells

| | Nevi | Melanoma | p-value (Wilcoxon |
|---|---|---|---|
| Cells | (n = 38) | (n = 34) | signed-rank test) |
| Binuclear and | 0% | 23% | <.001 |
| multinuclear | | (All ENV positive) | |
| ENV positive | 8% | 47% | <.001 |

Note:
Of the 34 melanoma specimens, 47% (16 cases) express ENV, and 23% (8 cases) have polyploid cells, and ALL the 8 cases with polyploid cells are ENV positive. However, 24% (47%-23%, 8 cases) cases are ENV positive but with no detectable polyploid cells, suggesting that ENV is necessary, but not sufficient, for the formation of polyploidy.

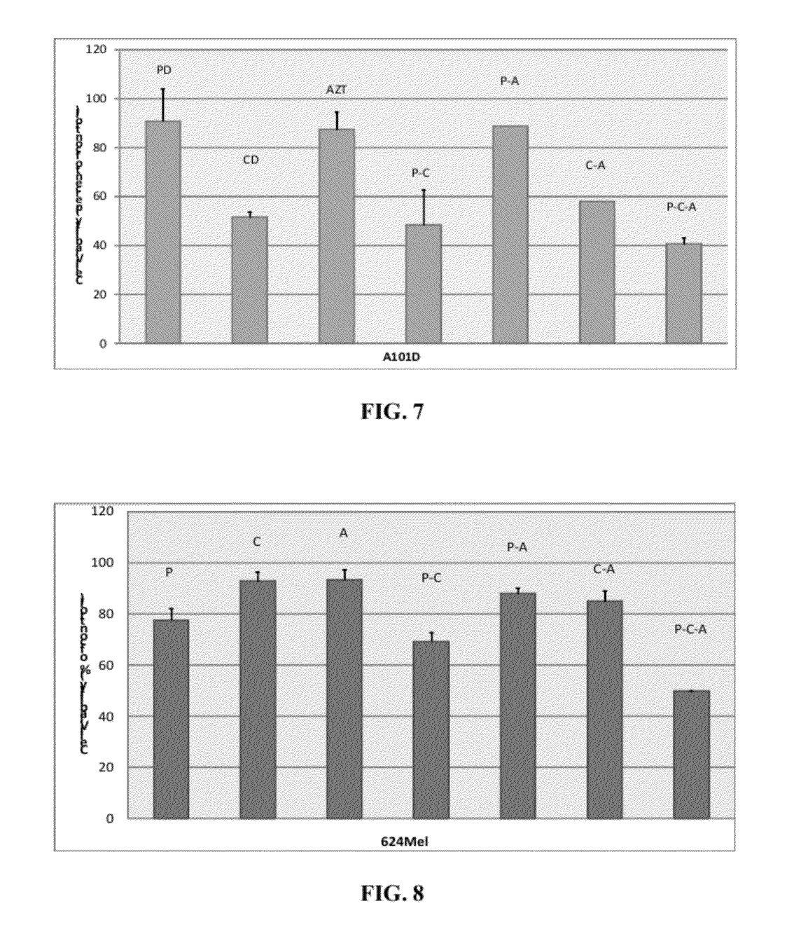

The inventors have simultaneously inhibited MEK and CDK4 using known pharmacological inhibitors PD98059 and 219476 along with using AZT to target HERV reverse transcription. See FIG. 7 and FIG. 8. Optimal results were obtained when the cells were treated for a period of 48 hours. A decrease in the number of viable cells was observed for both cell lines A101D and 624Mel. The data from this study provided preliminary data of the effect AZT has on melanoma cells in combination with inhibitors of MEK and CDK4, in support of the rationale for using AZT as a complementary treatment.

A101D and 624Mel human melanoma cell lines were grown in culture. CellTiter 96 Aqueous One Solution Cell Proliferation Assay (MTS, Promega) was used to measure total vial cells following manufacturer's instructions. 5,000 melanoma cells were seeded in each well of 96-well plates, and were grown for 24 hrs in media with 10% Fetal Bovine Serum (FBS). After that period, media were aspirated and various combinations of treatments were added to each well in media without serum. Based on dose-response analyses, the final concentrations used were: PD90895 at 25 μM; 219476 at 1 μM; AZT at 1000 μM. The plates were incubated for a period of 24 hrs, 48hrs, and 72 hrs, respectively. Then, the MTS solution was added to each well according to the manufacturers protocol. The plates were again incubated for 2-4 hrs, after which absorbance was measured at 490 nm. Data were analyzed and compared for treatment efficiency.

1. A method for treating melanoma comprising administering an effective amount of an anti-HERV therapy in combination with an effective amount of BRAF or MEK inhibitor, and a CDK4 inhibitor to a patient that has melanoma.

2. The method of claim 1, wherein the anti-HERV therapy is administered about 1, 2, 3, 4, 5, 6, 7, 8, 9, 10 hours or days prior to administration of the BRAF-MEK inhibitor and the CDK4 inhibitor.

3. The method of claim 1, wherein the BRAF or MEK inhibitor is PLX4032 (Plexxikon), BAY 43-9006 (Sorafenib), Raf-265 (CHIR-265), XL281 (Exelixis), dasatinib, erlotinib hydrochloride, gefitinib, imatinib mesilate, lapatinib, sorafenib tosylate, sunitinib malate, PD-325901, XL518, PD-184352, PD-318088, AZD6244, CI-1040, vemurafenib, GSK2118436, or combinations thereof.

4. The method of claim 1, wherein the BRAF or MEK inhibitor is PLX4032 or GSK2118436.

5. The method of claim 1, wherein the CDK4 inhibitor is P-276-00, GW-491619 (GlaxoSmithKine), AG-12275 (Pfizer), AG-12286 (Pfizer); PD-0166285 (Pfizer); PD-0332991 (Pfizer) or a combination thereof.

6. The method of claim 1, wherein the anti-HERV therapy is a monoclonal antibody and/or an anti-retroviral drug.

7. The method of claim 6, wherein the anti-HERV monoclonal antibody specifically binds a HERV envelope protein.

8. The method of claim 6, wherein the anti-retroviral is selected from a nucleoside reverse transcriptase inhibitors, non-nucleoside reverse transcriptase inhibitors, protease inhibitors, fusion inhibitors, or integrase inhibitors.

9. The method of claim 6, wherein the anti-retroviral is AZT, dideoxyinosine (ddI), dideoxycytidine (ddC), LAMIVUDINE (3TC), STAVUDINE (d4T), ABACAVIR (1592U89), ADEFOVIR DIPIVOXIL (bis(POM)-PMEA), NEVIRAPINE (BI-RG-587), DELAVIRDINE (BHAP, U-90152), EFAVIRENZ (DMP 266), INDINAVIR (MK-639), RITONAVIR (ABT-538), SAQINAVIR (Ro-31-8959), NELFINAVIR (AG-1343), AMPRENAVIR (141W94), or combinations thereof

10. The method of claim 1, wherein the melanoma is premalignant, malignant, metastatic, or drug-resistant melanoma.

11. The method of claim 1, wherein the patient is determined to be resistant to therapy.

12. An anti-cancer composition comprising an anti-retroviral, a BRAF or MEK inhibitor, and a CDK4 inhibitor.

13. An anti-cancer composition comprising an anti-HERV monoclonal antibody, a BRAF or MEK inhibitor, and a CDK4 inhibitor.

14. A method of treating melanoma comprising administering a pharmaceutically effective amount of an anti-HERV monoclonal antibody in combination with one or more of:
a) a BRAF or MEK inhibitor;
b) a CDK4 inhibitor; or c) a CTLA-4 inhibitor.

15. The method of claim 14, comprising administering an anti-HERV monoclonal antibody and PLX4032 or GSK2118436.

16. The method of claim 14, comprising administering an anti-HERV monoclonal antibody and ipilimumab.
